(19) United States
(12) Patent Application Publication
(10) Pub. No.: US 2016/0051027 A1
(43) Pub. Date: Feb. 25, 2016

(54) SYSTEM AND METHOD FOR ELIMINATING LICE AND NITS

(71) Applicant: Pam Skinner, Huntington Beach, CA (US)

(72) Inventor: Pam Skinner, Huntington Beach, CA (US)

(21) Appl. No.: 14/828,412

(22) Filed: Aug. 17, 2015

Related U.S. Application Data
(60) Provisional application No. 62/039,257, filed on Aug. 19, 2014.

(51) Int. Cl. A45D 24/30; A61F 7/00; A01N 63/00

(52) U.S. Cl. CPC A45D 24/30; A01N 63/00; A61F 7/00; A61F 2007/0008; A61F 2007/006; A61F 2007/0087

(57) ABSTRACT

A heated air system may include a heater/blower device, hose, and nozzle for blowing and heating air. The nozzle may be adapted and shaped for treatment of an ectoparasite infestation on an animal or person. Methods of treatment of an ectoparasite infestation may include applying a product to the hair to kill or slow hatched ectoparasites, combing out all the hatched ectoparasites, and heating air and applying heated air to a portion of the animal or person's body being treated to dehydrate any eggs and/or ectoparasites on the body.

### PRIORITY

This application claims the benefit of priority to U.S. Provisional Application No. 62/039,257, filed Aug. 19, 2014, which is incorporated by reference in its entirety into this application.

### BACKGROUND

Ectoparasites, including lice, can infest and feed on human hosts and cause various problems for the host, including discomfort, irritation, and itching. Head lice are insects that infest the heads of humans and grow to about the size of a sesame seed. Body lice infestations with some types of lice also occur.

Head lice infestations involve the colonization of the head or scalp of a host by the lice. Head lice feed on blood from the scalp and can cause irritation and itching for the host. Head lice are often spread to new hosts through direct head-to-head contact or sharing hairbrushes with an infested person. Head lice infestations are a significant problem with especially school age children, and many schools require children to leave the school until the infestation has been eliminated. School nurses often inspect children returning to school after treating a lice infestation. In schools with “no-nit policies” a child with any nits (viable or not) may not be allowed to return to school. This can result in negative consequences for children who are forced to miss important instruction in class. For these reasons, there needs to be a system and method which both kills and eliminates all lice and eggs from the scalp, in order to improve the industry standards.

Current solutions for treating ectoparasite infestations and/or head lice infestations are incomplete in their ability to fully eliminate the infestation and have other associated problems. For example, lice lay eggs or “nits” in the hair of the host. A nymph may hatch from each egg or “nit” and grow and develop to an adult louse. While existing treatments can be effective at killing nymphs and adult lice, these methods are often less effective at killing nits. Accordingly, even after a host receives a treatment that kills the adult lice and nymphs on a host's scalp, the lice infestation may return within a week when the surviving nits begin to hatch.

Pesticide based shampoos are available without a prescription, but these have multiple problems, for example, they are toxic with significant potential for harmful side effects for the animal or person to be treated. Also, shampoos, topical treatments, and/or home remedies can be ineffective at killing or eliminating eggs/nits, leaving the eggs/nits to hatch at a later time leading to re-infestation of the host. Many lice combs are poor quality (e.g., break easily) and are ineffective (e.g., comb teeth are spaced too far apart). Further, current treatments are very time consuming and may not remove adult lice, e.g., adult lice can sense motion and move quickly to a different section of the scalp as a comb passes through the hair thereby escaping the comb.

A heated air system called AirAllé™ uses heated air to treat lice. However, the AirAllé™ device is limited. Among other things, for example, the air flow volume is set to a specific, non-adjustable air flow rate. However, in terms of dehydrating lice air flow volume and the ability to adjust the air flow volume may be beneficial. For example, on very thick hair, higher air flow volume may be more effective at dehydrating and treating lice infestation. Having no option to adjust the air flow volume for individual persons can be limiting. Also, the AirAllé™ device does not offer options or controls for adjusting the temperature, operating exclusively at a temperature that does not vary more than a couple of degrees. However, higher heat might be desirable in certain circumstances and lower heat might be desirable in certain circumstances and for certain customer sensitivities. Additionally, having the option to adjust the temperature would provide the care provider more options in treatment, for example, higher temperature might be used for serious infestations or infestations including adult lice, while lower temperatures might be used for highly heat-sensitive individuals or when it is only necessary to dehydrate eggs or “nits” (e.g., after manual removal of adult lice and nymphs). Another limitation of the AirAllé™ device is that it is primarily focused on heat treatment, and does not provide for varied or combined treatment methods/systems that expand beyond heat treatment. Additionally, the nozzle of the AirAllé™ device is not optimal and can be difficult to use on the heads of persons. There is a need for an improved nozzle design that is easier for the treatment provider to use and that is more effective and efficient at treating the person.

Accordingly, there is an ongoing need for a reliable and safe systems, devices, and methods for treating ectoparasite infestations, including head lice infestations, that effectively kill eggs and/or hatched ectoparasites (e.g., ectoparasites in adult and nymph life stages). Successfully killing eggs, adults, and nymphs or successfully removing adults and nymphs and dehydrating eggs helps ensure that the ectoparasite infestation is eliminated and does not return. Effective and reliable treatment can also reduce the number of days that children are required to miss school due to ectoparasite infestation.

Further, it is desirable to have a system, device/apparatus, method, etc. that comfortably and quickly dehydrates lice and nits, which is affordable and portable, with a stand on wheels for easy movement. Also, it is desirable to have a system, device/apparatus, method, etc. that operates in a comfortable, but effective heat range, e.g., in a range from 115° F. to 127° F. or approximately this range. Still further, it would be desirable to have a system, device/apparatus, method, etc. that is more customizable with respect to air flow and operates with a variable air flow volume/rate control (e.g., which air flow may be measured in cubic feet per minute or “ft³/min” or “cfm”) as different hair thicknesses may be better treated at different air flow volumes, e.g., thicker hair benefits from higher cfm. It is also desirable to have a nozzle that is more comfortable to the person and the treatment provider and that is more effective. Therefore, there currently exists a need in the industry for systems, devices/apparatuses, methods, etc. that more effectively, efficiently, and comfortably eliminates lice and nits.

The present disclosure provides improved systems, devices/apparatuses, methods, etc. for treating ectoparasite infestations that are effective, comfortable, and affordable.

### SUMMARY

Embodiments of, and enhancements for, systems, devices/apparatuses, methods, etc. used for treatment of ectoparasite infestations, generally, and head lice infestations, in particular, are described herein.

According to various embodiments, the present disclosure provides a system including a heated, forced air system with a specially designed nozzle for killing ectoparasites or lice and a method associated with the system. In one embodiment, a high volume, heated forced air device is used. The device may be used to apply heated, forced air to the location of the infestation to dehydrate lice and nits at the location, thereby killing the lice and nits. The system includes a novel nozzle designed specifically for treating an ectoparasite infestation using a heated, forced air system to dry out and kill eggs and ectoparasites.

The systems, devices/apparatuses described herein may include: (1) a lower heat setting of 115° F.-127° F., (2) a lower air flow volume/rate range (10 cfm-23 cfm) (may be adjustable), and (3) an adjustable metal stand and mount, with wheels. The systems, devices/apparatuses described herein may include: (1) a higher heat setting of 160 f-180 f, (2) a higher air flow volume/rate range (135 cfm-150 cfm) (may be adjustable), and (3) an adjustable metal stand and mount, with wheels. The systems, devices/apparatuses described herein may include: (1) controls for adjustment to different heat settings, e.g., a range of low, medium, or high heat settings, (2) controls for adjustment to different air flow volume/rate settings (e.g., from 5 cfm-250 cfm), and (3) an adjustable metal stand and mount, with wheels.

The methods described herein may include (1) a specific technique to section and treat the scalp of a person with infested with head lice, (2) the application of a non-toxic scalp treatment with the utilization of a micro-grooved nit comb, and (3) a specific method of “nit-picking” to remove all lice and eggs using magnification, bright light, and a method of parting hair.

According to one embodiment a method of treating an ectoparasite (e.g., head lice) infestation to kill the nits or eggs, adult ectoparasites, and any ectoparasites in other life stages (e.g., nymph) includes first sectioning the hair into sections (hair clips, ties, bands, etc. may be used to separate and keep separate the sections), e.g., into 3-4 sections. Next, each section of hair is sequentially unclipped/released and may be held in one hand while the other hand is used to run a nozzle of the device over and/or through the isolated section of hair to apply heated, forced air to the hair and/or scalp through the nozzle. The nozzle may be held in a first location/area of a first section for a certain amount of time effective to kill the ectoparasite or lice without overheating or over stimulating the scalp, e.g., 15 seconds to 5 minutes, 25 seconds to 120 seconds, 30 seconds to 90 second, 60 seconds or 30 seconds. After applying the heated air to the first location/area of the first section for the desired time, the nozzle may be moved approximately 3 inches to a second location of the first section that overlaps the first location/area, e.g., by 50%. The heated air is applied through the nozzle to the second location/area for a similar amount of time, e.g., a 30 to 90 second interval or other time interval described above. This process is continued until the entire first section of hair has been treated with heated air. The first section may be re-clipped, tied, or otherwise held isolated from the other sections. Then the second section of hair may be unclipped/released, and the steps described above of heating sequential locations/areas of the first section are likewise applied to the second section. These steps may be repeated for all the isolated sections of hair, until the entire scalp has been treated with heated air as described.

Before and/or after the heated, forced air treatment, a product (e.g., shampoo, spray, or other topical product) may be applied to the hair and/or scalp to kill or “slow down” any remaining lice or nits. The product used is preferably non-toxic. Then the hair in each section may be thoroughly combed out until no nits or lice remain (e.g., no more than 1.5% to 0% remain) on the head. A microgrooved metal nit comb may be used. After combing, one may optionally do “nit picking,” i.e., going through the entire head in very thin sections doing a visual check for any remaining nits, and pulling them (e.g., one by one) off the hair. Magnification may be used to facilitate this process. Ultimately, at the conclusion of these steps, the scalp is generally totally free of all visible lice and nits. Any very small, microscopic, or missed lice or nits are killed/rendered nonviable by the steps above (e.g., via dehydration during air treatment), and the infested scalp will be completely or 100% free of viable lice and nits.

In one embodiment, a method of treating a person may comprise applying a topical product to hair of an animal or person to kill or slow down any hatched ectoparasites on the head; dividing hair of a person into multiple isolated sections; and combing a first isolated section of hair of the multiple isolated sections with a comb until all hatched ectoparasites (e.g., 99% or more) are removed from the first isolated section of hair. The combing may also be done to the other isolated sections of the multiple isolated sections before any application of heated air or sequentially as each section is treated with heated air. The method may include applying heated air through a nozzle of a heated air system to the first isolated section of hair at a temperature and air flow sufficient to dehydrate any eggs in the first isolated section. Optionally, the method may include picking though the first isolated section in very thin subsections of hair doing a visual check for any remaining eggs and, if found, pulling the remaining eggs off the hair. The method may also comprise combing a second isolated section of hair of the multiple isolated sections with the comb until all hatched ectoparasites are removed from the second isolated section of hair; and applying heated air through the nozzle of the heated air system to the second isolated section of hair at a temperature and air flow sufficient to dehydrate any eggs in the second isolated section. Optionally, the method may also include picking though the second isolated section in very thin subsections (e.g., paper-thin subsections) of hair doing a visual check for any remaining eggs and, if found, pulling the remaining eggs off the hair.

The method may also include combing a third isolated section of hair of the multiple isolated sections with the comb until all hatched ectoparasites (e.g., 99% or more) are removed from the third isolated section of hair; and applying heated air through the nozzle of the heated air system to the third isolated section of hair at a temperature and air flow sufficient to dehydrate any eggs in the third isolated section. Optionally, the method may include picking though the third isolated section in very thin subsections of hair doing a visual check for any remaining eggs and, if found, pulling the remaining eggs off the hair. The method may also include combing a fourth isolated section of hair of the multiple isolated sections with the comb until all hatched ectoparasites (e.g., 90% or more) are removed from the fourth isolated section of hair; and applying heated air through the nozzle of the heated air system to the fourth isolated section of hair at a temperature and air flow sufficient to dehydrate any eggs in the fourth isolated section. Optionally, the method may also include picking though the fourth isolated section in very thin subsections of hair doing a visual check for any remaining ectoparasites or eggs and, if found, pulling the remaining eggs off the hair.

Applying heated air through the nozzle of the heated air system to the first isolated section of hair or to the other isolated sections of hair may include applying the heated air to a first area within the first isolated section for first time interval sufficient to dehydrate any eggs in the first area, then moving the nozzle to a second area within the first isolated section, wherein the second area overlaps the first area, and then applying the heated air to the second area for second time interval sufficient to dehydrate any eggs in the second area. The first time interval and the second time interval may each be between 30 and 90 seconds, between 50 and 70 seconds, between 30 and 60 seconds, about 30 seconds (e.g., within a second or two), about 60 seconds (e.g., within a second or two). The second area may overlap the first area by between 25-65%, by 45-55%, or by other percentages. Applying heated air through the nozzle of the heated air system to the first isolated section of hair or the other isolated sections of the multiple isolated sections may further comprise moving the nozzle to a third area within the first isolated section, wherein the third area overlaps the second area, and then applying the heated air to the third area for a time sufficient to dehydrate any eggs in the third area. The time intervals and amounts of overlap involving the third area may be the same or similar to those above.

Applying heated air through the nozzle of the heated air system to the first isolated section of hair or other isolated sections of the multiple isolated sections may also include applying the heated air sequentially to multiple areas within the first isolated section for a time sufficient to dehydrate any eggs in each of the multiple areas until the first isolated section has been entirely treated with heated air, and wherein adjacent areas overlap. When applying heated air through the nozzle of the heated air system to an isolated section of hair, the nozzle may be held a majority of the time at about 0.15 to 0.35 inches, 0.25 to 2 inches, 0.5 to 1.5 inches, 1 to 1.5 inches, or other distances away from the scalp. When applying heated air through the nozzle of the heated air system to one or more of the isolated sections of hair, hair may be allowed to “parachute” or expand allowing the heated air to be directed through the hair, to the base of the hair. Optionally, the hair may be pulled to the side and not allowed to “parachute.”

The nozzle of the heated air system may be comb-shaped. The nozzle of the heated air system may be kidney shaped and have a concave treatment end. The nozzle may include extending portions/extensions/teeth that extend from the treatment end and can pass through and separate portions of a person's hair. The extending portions/extensions/teeth may be removably attachable to the nozzle. The nozzle may include a gripping member on a back end of the nozzle, the back end being opposite the treatment end.

Furthermore, the processes or methods described herein may include: (1) combining a method of dehydrating ectoparasites (e.g., lice) and eggs with a high volume of heated air, using a non-toxic product (e.g., mousse, foam, spray, shampoo), nit combing, and nit-picking to ensure that 100% of ectoparasites and/or eggs are killed/non-viable, (2) a specific methodology of sectioning, combing or spraying and combing, and removing all ectoparasites and eggs (e.g., lice and nits) from the hair, and (3) heat (to dehydrate), non-toxic head treatment (to kill or slow hatched lice, and a specific methodology of sectioning, combing, and removing all lice. Therefore, the disclosed methods and associated systems/devices, etc. create one seamless process which will have an extremely high success rate for elimination and removal of lice and eggs on the scalp.

### BRIEF DESCRIPTION OF THE DRAWINGS

The disclosed systems, devices/apparatuses, applications, methods, etc. can be better understood with reference to the following drawings.

FIG. 1 shows a perspective view of an exemplary heated, forced air system that may be used for treating an ectoparasite infestation to dry out and kill eggs and/or ectoparasites.

FIG. 2 shows an exploded view of the exemplary heater/blower device of the heated, forced air system of FIG. 1 that shows component parts of the device.

FIG. 3 shows another exemplary heated, forced air system that may be used for treating an ectoparasite infestation to dry out and kill eggs and/or ectoparasites or that may be used to perform the methods/treatments described herein.

FIG. 4A shows a side view of an exemplary nozzle that may be used with the heated, forced air systems described herein for treating an ectoparasite infestation, e.g., to dry out and kill eggs and/or ectoparasites.

FIG. 4B shows a view of an exemplary nozzle similar to the nozzle of FIG. 4A looking down the extensions or teeth of the nozzle.

FIG. 5A shows a perspective view of a novel nozzle designed specifically for treating an ectoparasite infestation using a heated, forced air system to dry out and kill eggs and/or ectoparasites.

FIG. 5B shows front side view of the nozzle of FIG. 5A.

FIG. 5C shows top view of the nozzle of FIG. 5A.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but rather the intention is to cover all modifications, equivalents, and alternatives falling within the spirit and scope of the invention as defined by the appended claims.

### DETAILED DESCRIPTION

The following description and accompanying figures, which describe and show certain embodiments, are made to demonstrate, in a non-limiting manner, possible configurations and steps of systems, devices/apparatuses, and methods for the treatment and elimination of ectoparasite infestations and/or eggs left by ectoparasites. While the description herein, by way of example, is focused primarily on a description of treating head lice infestations in humans, the systems, devices/apparatuses, methods, etc. described herein are not so limited and may be applied to treating other types of insect infestations or ectoparasite infestations or other types of infestations that do not involve lice. Further, while infestations of the head are primarily discussed herein, the systems, devices/apparatuses, methods, etc. described herein are not so limited and may be applied to infestations in areas other than a host's head. Infestations that may be treated with the systems, devices/apparatuses, methods, etc. described herein may be on human hosts, on animal hosts, or may be infestations in a variety of other locations. Accordingly, the inventive principles associated with the embodiments described herein, may be applied to other embodiments and other types of infestations, systems, devices/apparatuses, methods, etc.

According to various embodiments, the objectives described above and elsewhere herein may be accomplished by providing an improved system for killing ectoparasites or lice and treating ectoparasite or lice infestations. The present disclosure provides a system including a device for killing ectoparasites or lice and/or their eggs and a method associated with the system. The system may include a specially designed nozzle for use in killing ectoparasites on a body or part of a body (e.g., a head). In one embodiment, the heated, forced air system is easily portable, electrically powered (and/or battery powered), and can be used to direct controlled heated airflow to dehydrate lice and eggs on the head and scalp of people with head lice infestations.

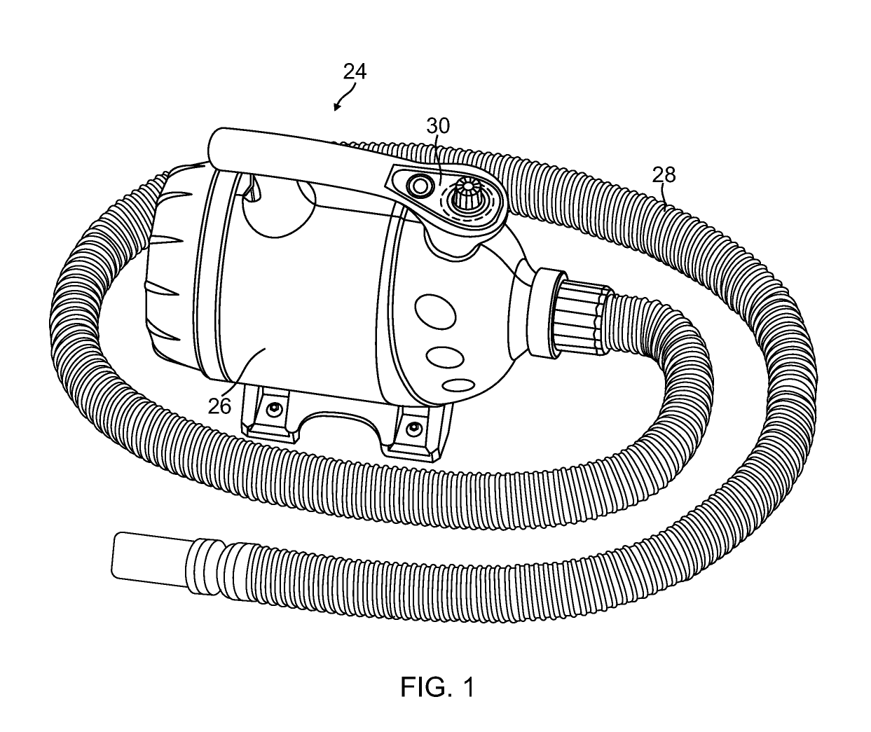

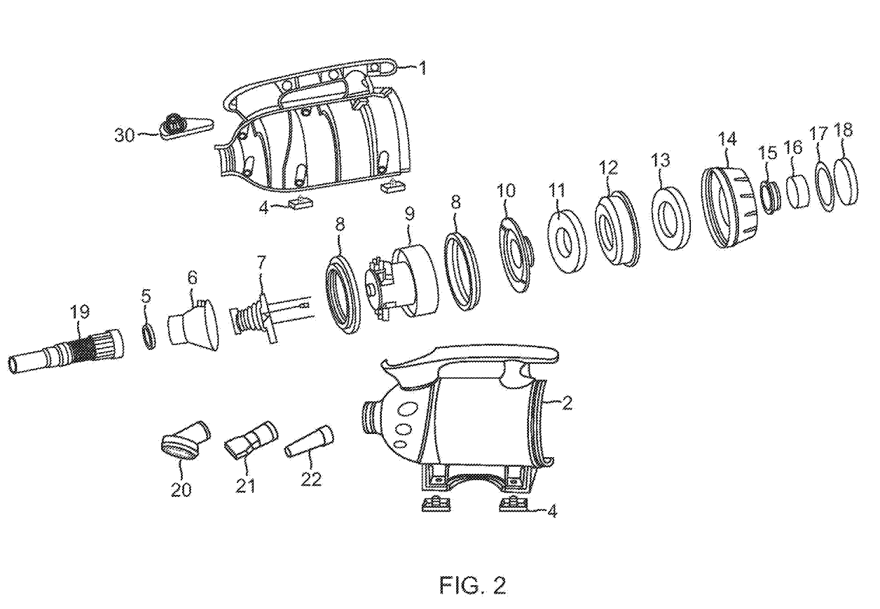

A high-volume, heated, forced air system 24 such as that shown in FIGS. 1 and 2 may be used with the specially designed nozzle and methods described herein. FIG. 1 shows a perspective view of an exemplary heated, forced air system 24 that may be used as described herein. The heated, forced air system 24 may include a heater/blower main unit 26, a hose 28 (e.g., an 8 foot flexible, heat protected nylon hose) attached to the heater/blower 26, and a nozzle (e.g., one or more of nozzles 20, 21, 22, 120, 220). The heater/blower device 26 may include a handle for lifting and moving the device and a control panel 30 for adjusting various settings (e.g., temperature, air flow, anion, etc.). The control panel 30 may include a variety of controls, knobs, switches, etc., e.g., a knob rotatable to adjust air flow volume/rate or cfm, a knob rotatable to adjust temperature settings, a switch to turn on or off anion compensation to reduce static). The heated, forced air system 24 may be used to dehydrate lice and nits on the head, thereby killing lice and nits both or just nits. The device can be used, for example, in a 25-90 second interval, a 30 second interval, or a 1 minute interval, to dehydrate all nits and lice on the head, and render them nonviable.

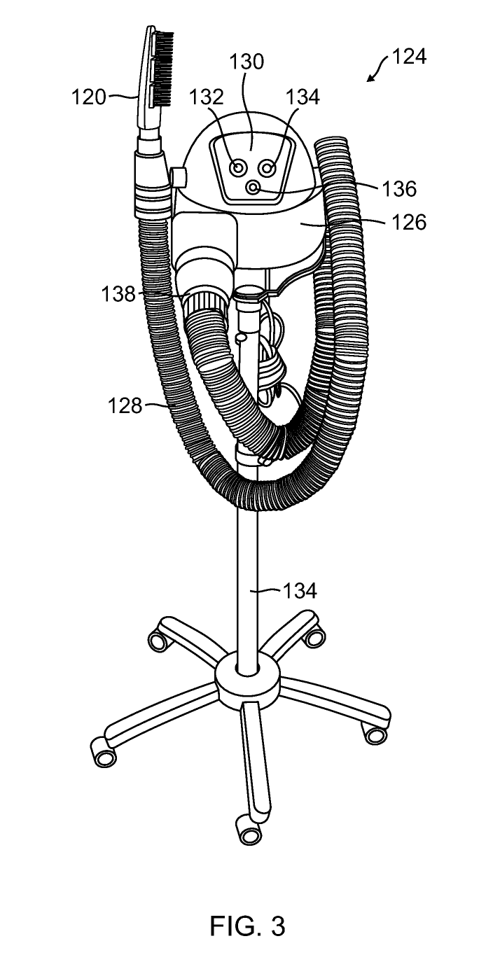

A variety of heater/blower devices or units may be used in the heated, forced air system 24, including the heater/blower device 126 of FIG. 3 or other heater/blower devices. The various heater/blower devices that might be used may comprise a variety of components and parts. For example, FIG. 2 shows an expanded view of the device in FIG. 1 with various exemplary parts and components of the device shown. Components of the various heater/blower devices may include a housing 1, 2 (e.g., a durable ABS housing), a motor or pump 9 (e.g., a 3 HP, 14 amp motor), a hose 19, a nozzle (e.g., nozzles 20, 21, 22, 120, 220) for directing the heated, forced air at the hair or scalp to treat the infestation (e.g., a durable plastic nozzle that may include extension 140, 240 to comb through and separate the hair during treatment), a stand mount, an electrical cord (e.g., a 10 foot electrical cord for a 3-prong GFCI outlet), a battery (e.g., for more mobile, unplugged use). The heater/blower device(s) may also include other components/part, including a left housing 1, a right housing 2, a control panel 30, rubber feet 4, air outlet grid 5, heater element round cover 6, heater element 7, motor rubber sleeve 8, motor 9, dustproof cover 10, motor filter 11, dust case 12, dust case filter 13, rear cover 14, rear cover connector 15, crossed cover 16, rear cover baffle 17, inlet filter 18, hose 19, round groomer rake nozzle 20, flat nozzle 21, cone nozzle 22, etc. Wheels or a stand (e.g., stand 134) with wheels may also be included. The heated, forced air system is preferably portable and may include a handle for carrying and/or wheels for rolling the device around.

The heated, forced air system may include a variable air flow volume/rate control or knob that can adjust air flow volume/rate (e.g., between 10 to 200 cfm, between 5 to 23 cfm, between 135 to 150 cfm, between 10 to 100 cfm, etc.) and variable temperature control/knob/switch that can adjust temperature (e.g., between 100° F. to 200° F., between 110° F. to 127° F., between 160° F. to 180° F.), so the treatment may be customized to particularly benefit individual animals or persons. Alternatively, the temperature may be set to specific temperature or narrow range of temperatures that may not be variable by the user or may only be slightly variable in a narrow range, e.g., at a temperature between 110° F. and 127° F., between 115° F. and 125° F., at 125° F., at 120° F., between 160° F. to 180° F., or between 160° F. or 170° F. A thermometer (e.g., a thermocouple or other temperature measuring device) may be used with the system to ensure that the desired temperature has been reached before applying any heated air to the animal or person, this helps reduce the amount of time that heated air must be applied to the animal or person and reduces the likelihood that a section or area of the head may be treated at a temperature too low to be effective.

The systems, devices/apparatuses discussed herein may include an optional portable stand mount (e.g., stand 134) with wheels. This makes it much easier for the operator to position the device so that the digital timer can be easily viewed, also so that the hose (e.g., an 8 foot, heat protected, nylon hose (note other types of hoses and other lengths may be used)) is positioned safely, so as not to create a tripping hazard.

A variety of nozzles may be used with the heated, forced air system, including any of the nozzles described herein. The heated, forced air system preferably including an interface for quickly removing and attaching a variety of different nozzles so treatment can be customized for particular animals or persons for treating different areas of the animal's or person's body and/or head. The interface may be on a hose or the device itself and may be designed to attach to the nozzle by friction fit, complimentary locking portions, latches, threading, etc. FIGS. 2-5C show some nozzles that may be used with the devices and in the methods described herein. FIG. 2 shows three nozzle types at 20, 21, and 22 that may be used. In one embodiment, a nozzle with extensions circling around a central opening may be used to help separate the hair during treatment. Other nozzle options, designs, and sizes are also possible. For example, any of the nozzles described herein may be made in a variety of sizes, (e.g. a small size nozzle for smaller heads, and a large size nozzle for large heads, etc.). Other nozzle options, designs, and sizes are also possible. For example, any of the nozzles described herein may be made in a variety of sizes, (e.g. a small size nozzle for smaller heads, and a large size nozzle for large heads, etc.).

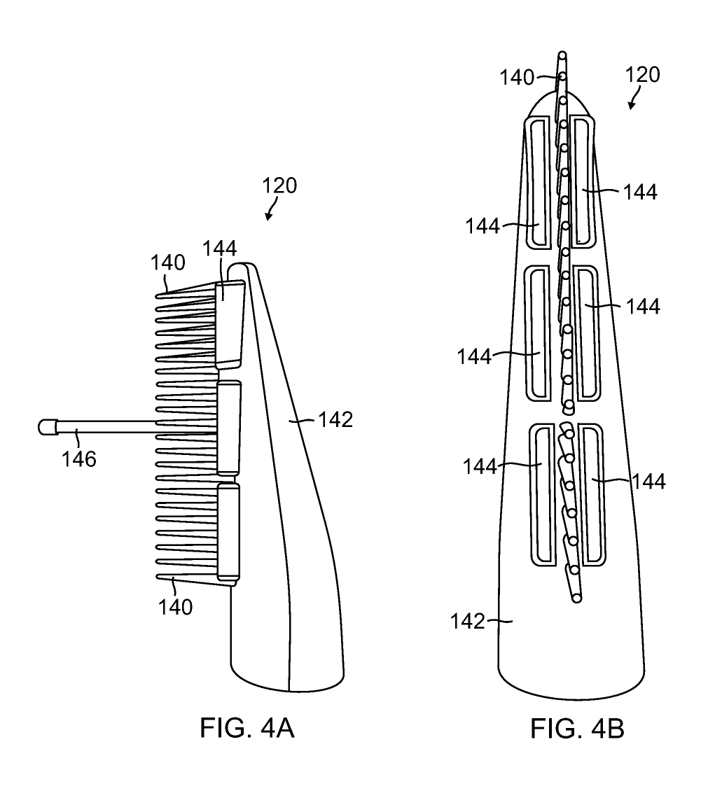

FIG. 4A shows an exemplary nozzle 120 that may be used with the forced, heated air systems described herein. FIG. 4A shows a comb-like nozzle with teeth or extensions 140 extending perpendicular to a center line or longitudinal axis of the nozzle. The extensions are shown as being arranged along a straight line with each extension 140 parallel to each other; however, other arrangements for the teeth or extensions 140 are also possible. The nozzle may include one or more openings 144 on either side of the extensions 140. In one embodiment, there may be three openings 144 on either side of the extensions, e.g., as shown in FIG. 4B, but other numbers of openings are also possible, e.g., 1, 2, 3, 4, 5, etc. The openings 144 may be a variety of shapes, e.g., square, rectangle, circular, ovoid, triangular, pentagonal, hexagonal, etc. The nozzle may include a main portion or grip portion 142 from which the extensions 140 extend. The main portion or grip portion 142 may include a passageway or lumen therein into which the openings 144 open to allow passage of air through the passageway and out of the openings 144 from the heater/blower device. Optionally, the nozzle 120 may include an extender 146 configured to keep the extensions 140 a certain distance (e.g., 2 inches, 1.5 inches, 1 inch, 0.5 inches, any sizes between these, etc.) from the body or head being treated during use. Other nozzles used with the system, even if different from nozzle 120, may use an extender as well. FIG. 4B shows a view of nozzle the same as or similar to the nozzle of FIG. 4A, but without an extender 146. FIG. 4B looks down the extensions 140 toward the main portion or grip portion 142 and openings 144. The main portion or grip portion 142 may include one or more gripping features (e.g., loops/rings for fingers, textured surface, ridges, ridges and valleys configured to match fingers, a non-slip surface, gripping members similar to those discussed below, etc.) to make holding and manipulating the nozzle easier.

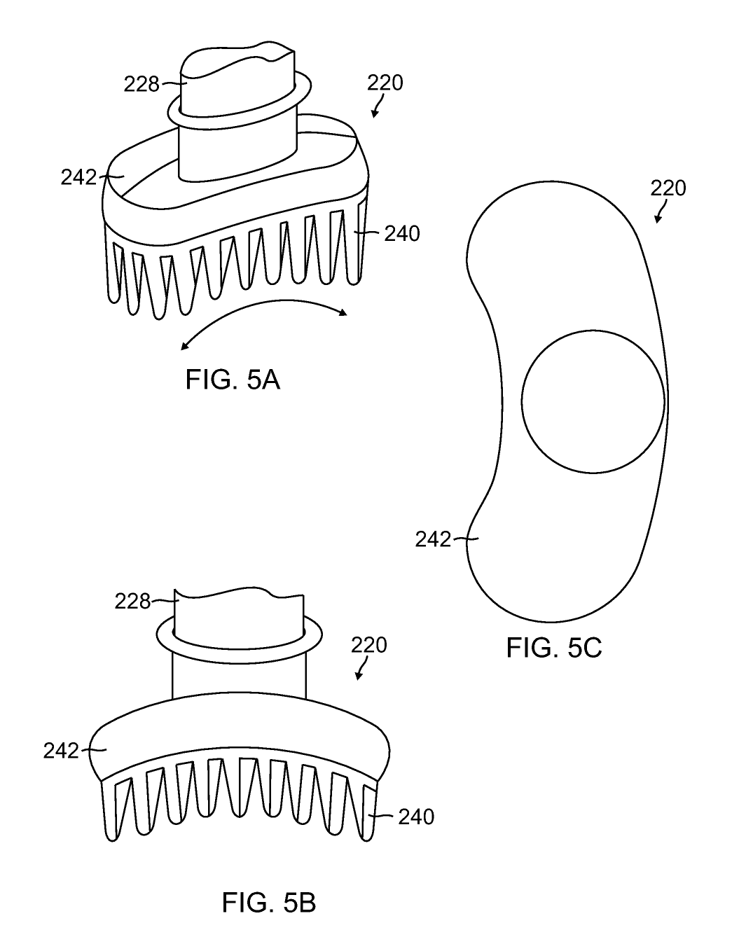

FIGS. 5A-5C show different views of a significantly improved nozzle 220 that makes providing heated air treatment and dehydrating lice easier, more effective, and more efficient. The nozzle is easier for a treatment provider to use and is more comfortable for the animal or person being treated, as well as being more effective for treating ectoparasite infestations on a rounded surface (e.g., a human head). As shown in FIGS. 5A and 5B, the treatment end of the nozzle is concave so that it better fits the curve of the rounded surface or the curve of a person's head. This can help better direct the heated air to the desired treatment area/location, even when the treatment area/location includes, for example, a sloping or curved portion of the head. The nozzle may be sized and/or configured to fit or work most effectively with the average human head or to fit or work most effectively with particular head sizes (e.g., a small head size for children, a larger size for adults). Other shapes may be sized/designed/configured to fit better with other animals.

Also, the treatment end in FIGS. 5A and 5B is shown with a series of extending portions or extensions 240 (e.g., teeth or jagged teeth) extending from the treatment end of the main portion or grip portion 242 of the nozzle 220 around the opening of the nozzle 220 through which the heated air passes, but other types of extending portions or parts designed to comb through or separate portions of the hair may also be used (e.g., bristles). The extending portions 240 (e.g., bristles, teeth, etc.) of the nozzle 240 help brush through and separate the hair during treatment. The extending portions 240 (e.g., bristles, teeth, etc.) can be stiff and rigid which is beneficial in certain applications. Optionally, the extending portions 240 may be softer and/or more flexible for added comfort to the person being treated, or a combination of flexible softer extending portions, bristles, or teeth (e.g., made of silicone) and more rigid extending portions, bristles, or teeth may be used. However, the extending portions 240 (e.g., bristles, teeth, etc.) may be adequate to lift and/or separate the hair in a way to allow maximum exposure by the heated air and prevent ectoparasites or lice from hiding from the heated air in a clump of hair. The extending portions 240 can surround the opening in the nozzle without extending over the opening, or may be designed to extend over the opening as long as air may still flow out of the opening. In one embodiment, beams may extend over the opening (whether in series of parallel lines or a crisscrossing fashion), the beams including extending portions, bristles, or teeth thereon, but leaving openings between the beams through which the heated air may pass. Similarly a mesh-like network with extending portions, bristles, or teeth may extend over the nozzle opening as long as air may pass through the smaller openings in the mesh-like network.

Optionally, a nozzle shaped like the nozzle 220 in FIGS. 5A-5C may be provided without permanently attached extending portions, bristles, or teeth. The nozzle may be used without any extending portions, bristles, or teeth. Optionally, the nozzle itself may be provided with various removably-attachable attachments with extending portions, bristles, teeth, and/or different shaped openings. The removably-attachable attachments may be attached to the treatment end of the nozzle to customize the nozzle for particular persons being treated or types of hair (e.g., thicker hair vs. thinner hair). The extending portions, bristles, or teeth may be in similar configurations to those discussed above, but be attached to the removably-attachable attachments.

As shown in FIG. 5C, the nozzle 220 is generally kidney-shaped or bean-shaped, which makes it easier for the treatment provider to use. The nozzle 220 may be of various sizes, e.g., it may be sized such that the treatment portion of the nozzle extends wider than shown in FIG. 5C and extends further beyond the cylindrical hose attachment. A treatment provider may grip the nozzle 240 around the tubular shaped portion 244 with a thumb and forefinger and allow the remaining fingers and the rest of the hand to cup around the back of the nozzle, which further helps to direct the air to the areas desired and is more comfortable during use. The hose (e.g., hose 28, 128) of the heated/forced air system may attach to the nozzle through the tubular shaped portion 244 extending from a surface of the main portion or grip portion 242, the surface being a back surface opposite the treatment end or a side surface.

The nozzle 220 may also be contoured to fit comfortably with a human hand to allow for greater comfort of the treatment provider and easier gripping of the nozzle. Additionally, one or more gripping features (e.g., similar to those discussed above) or gripping members, e.g., an extension(s), strap(s), hook(s), loop(s), etc., may be located on the back of the nozzle (i.e., opposite the treatment end) to allow for easier gripping of the nozzle or fastening of the nozzle on the treatment provider's hand. For example, a hook-like extension may project/extend from the back of the nozzle that fits between two fingers of the treatment provider and hooks partially over the top of the hand. Or a series of ring-like loops (e.g., 2, 3, 4, or 5 loops) may extend from the back of the nozzle through which one or more fingers of the treatment provider may be placed when gripping the nozzle. The loops may be of various sizes, thicknesses, and lengths.

The nozzle (e.g., any of the nozzles described herein) may be held in contact with the scalp or may be held above the scalp (e.g., 0.5 to 2 inches, 1 to 1.5 inches, 1 inch, 0.5 inches, or 1.5 inches above the scalp) during treatment. The nozzle may include a vibrating component that causes the nozzle and/or the extending portions, bristles, or teeth to vibrate to help separate the hair and/or to massage the scalp during treatment. The vibrating component may be separately turned on or off.

The system, devices/apparatuses, applications, methods, etc. discussed herein may be used to dehydrate and thereby render non-viable and eliminate eggs (e.g., “nits”) and/or dehydrate and kill ectoparasites (e.g., hatched ectoparasites, nymphs, lice, etc.). The following components/aspects/steps may be used: a heated forced air system (including heater/blower device producing heated air, hose, nozzle, stand, and/or other features described herein), a non-toxic lice treatment product (e.g., shampoo, mousse, or spray), a microgrooved, metal nit comb, and magnification (e.g., magnifying glass and or magnifying goggles) for final “nit picking” step. These components may be used together with the methods described to eliminate generally 100% of lice and nits off a single human head. In one embodiment, first, the entire scalp/hair is dehydrated using the dryer device, in 30 second, 45 second, or 1 minute intervals, overlapping treatment areas/locations over the entire scalp. This step may be especially effective on nits/eggs, as it dehydrates the amniotic fluid in the egg, and renders the eggs nonviable. Second, applying a non-toxic lice treatment product (e.g., a mixture of conditioner, and/or oil, and/or water, or other enzyme-based foams, mousses, shampoos or sprays) that is designed to kill and/or limit or prevent mobility of hatched lice (adults or nymphs (recently hatched lice). Third, sectioning (can be same sections as used in heated air treatments described herein or new sections) and combing the entire scalp with a micro-grooved nit comb, is designed to remove all lice (or other ectoparasites) and as many nits as possible from the scalp. Fourth, “nit-picking” (e.g., going through entire scalp, with magnification, in paper thin sections) is done to make sure that virtually all of lice and eggs/nits are removed from the hair and scalp. These steps can be performed in a different order as well, e.g., the second and third steps above may be performed before any heat treatment (i.e., before the first step) such that hatched ectoparasites and large eggs are removed before heat treatment and heat treatment primarily dehydrates smaller eggs left behind after the combing.

According to one embodiment a method of treating an ectoparasite (e.g., head lice) infestation to kill the nits or eggs, adult ectoparasites, and any ectoparasites in other life stages (e.g., nymph) includes first sectioning the hair into sections (hair clips, ties, bands, etc. may be used to separate and keep separate the sections). The hair may be sectioned into any number of sections, for example, in 2-10 sections, but more preferably into 3-4 sections. Next, each section of hair is sequentially unclipped/released and held in one hand while the other hand is used to run a nozzle of the device over and/or through the isolated section of hair. The system/device forces/blows heated air through the nozzle toward the hair and/or scalp. In one embodiment, the nozzle is held approximately ¼ inch to 2 inches, ½ inch to 1.5 inches, or about 1 inch or 1.5 inches off or away from the scalp. In one embodiment, the hair may be allowed to “parachute” or expand around the nozzle, allowing the heated air to be directed through the hair and/or to the base of the hair. In one embodiment, the hair is held to the side and not allowed to parachute. The nozzle may be held in a first location/area of a first section for a certain amount of time effective to dehydrate eggs and/or kill the ectoparasite or lice without overheating or over stimulating the head of the person being treated, e.g., 30 seconds to 90 seconds, 30 seconds, 45 seconds, or 60 seconds (or about these times, e.g., within 2 seconds of these). After applying the heated air to the first location/area of the first section for the desired time, the nozzle may be moved a distance (e.g., approximately 2-4 inches or 3 inches) to a second location of the first section. In one embodiment, the second location/area overlaps the first location/area, e.g., overlapping by 50%. The heated air is applied through the nozzle to the second location/area for a similar amount of time. This process is continued until the entire first section of hair has been treated with heated air. The first section may be re-clipped, tied, or otherwise held isolated from the other sections. Then the second section of hair may be unclipped/released, and the steps described above of heating sequential locations/areas of the first section are likewise applied to the second section. These steps are repeated for all the isolated sections of hair, until the entire scalp has been treated with heated air as described.

After the heated air treatment, a product (e.g., enzyme-based foam or mousse, or spray, or other topical product) may be applied to the hair and/or scalp to kill or “slow down” any remaining lice or nits. The product is preferably non-toxic. A non-toxic product can be specifically for lice (such as an enzyme-based or protease-based product), or a mixture of water and conditioner, or a non-toxic oil. Then the hair in each section may be thoroughly combed out until no nits or lice remain on the head. A microgrooved metal nit comb may be used. After combing, one may optionally do “nit picking,” i.e., going through the entire head in very thin sections doing a visual check for any remaining nits, and pulling them (one by one) off the hair. Magnification may be used to facilitate this process. Ultimately, at the conclusion of these steps, the scalp is generally totally free of all visible lice and nits. Any microscopic or missed lice or nits are killed/rendered nonviable by the steps above, and the infested scalp will be completely free of lice and nits.

Similarly, the method associated with the present invention may also include one or more of the following steps: The first optional step is referred to as “detection combing.” This involves misting a portion of the scalp (e.g., with water or with a spray designed to kill or slow the ectoparasites or lice; options include products including: protease based enzymes, dimethicone based products, or a mixture of products like olive oil, hair conditioner, and/or water, combined and poured into a spray bottle to mist or saturate hair), detangling hair with a hair brush, passing a nit comb (e.g., a microgrooved metal nit comb) through the section, and wiping both sides of the nit comb on a damp paper towel. If ectoparasites, nits, or lice appear on the paper towel, this indicates an infestation. Another method of “detection combing” is to leave the hair dry, detangle, pass the nit comb (e.g., microgrooved metal nit comb) through the hair, and “strum” the comb into a white ceramic bowl, causing any lice or nits to fall from the comb into the bowl, making them easily visible.

Various techniques may be used to section and comb hair. For example, the hair may be divided into a specific number of sections, and each section may be clipped with a hair clip (or other hair holding device). The hair may be divided into 2 sections, holding the hair as if you were going to make a “pony tail” (or two “pony tails” or “pig tails” one on each side of the head). Combing steps described herein may be done by passing the nit comb (e.g., microgrooved metal nit comb) from “root to tip” of hair, and continuing until no lice or nits come out. With respect to “nit-picking,” one may section the hair (e.g., into 4-6 sections), clip or tie each section, begin with the first and make vertical and/or horizontal parts (e.g., into a series of “paper thin” sections), and inspect the hair using magnification and bright, direct light (hands free magnifying visor and headlamp, or magnifying roller stand lamp may be used).

Treatment methods may include the following steps: First, section the hair into sections (e.g., 3, 4, 5, 6, or more sections) (use hair clips, ties, band, or other hair devices to separate). Second, hold each section of hair in one hand, and insert the nozzle through the hair (e.g., the nozzle or nozzle extensions/teeth may be held approximately ¼ inch, ½ inch, ¾ inch, 1 inch, 1.25 inches, 1.5 inches, or 2 inches off the scalp). The hair may be allowed to “parachute” around the nozzle, directing the air to the base of the hair. Optionally, the hair may be pulled to the side and inhibited or prevented from parachuting. The nozzle may be held in one location for a certain time (e.g., 60 seconds). Third, the nozzle may be moved approximately 2-4 inches, being careful to overlap previous area by 50%, and repeat the time interval (e.g., 30, 45, 60 seconds or other interval). This process is continued until the entire section of hair has been covered. The section may be re-clipped, and the next section unclipped. This process may be continued until all sections (e.g., all 3-4 sections of hair) and the entire scalp has been covered. In the fourth step, one may apply a non-toxic product to kill or “slow down” and remaining lice or nits. The non-toxic product can be a protease based product, or a mixture of water and conditioner, or a non-toxic oil. The Fifth step may include thoroughly combing out each section, until no nits or lice remain on the head, using a nit comb (e.g., a microgrooved metal nit comb). The last (optional) step is “nit picking,” going through the entire head, in very thin sections, using magnification, doing a visual check for any remaining nits, and pulling them (one by one) off the head. These steps may be performed in different orders as well, e.g., the fourth and fifth steps may be performed before the second and third steps to remove hatched ectoparasites or lice and large eggs manually, then dehydrate any remaining eggs.

It should be further noted that a “detection combing” step (e.g., passing a microgrooved metal nit comb through at least one or more sections of the scalp, and confirming the presence of lice or eggs) is a good idea before performing the other treatment steps.

In one embodiment, a method of treatment using a heated air system that heats air and may be manipulated to direct the heated air in such a way as to kill ectoparasites (e.g., lice) and eggs (e.g., lice eggs or nits) on a person is used. One method may exclusively involve treating persons with the heated air system at temperatures of 150° F. or higher, 155° F. or higher, or 160° F. or higher with a flow rate of 130 cfm or higher or 135 cfm or higher, as this may effectively kill both lice and lice eggs. Higher air temperatures and/or higher cfm may be more effective at killing hatched ectoparasites. Lower temperatures may still be effective against hatched ectoparasites, but may be especially effective for dehydrating and rendering non-viable eggs or nits. Before any heated air is applied to any portion of an animal's or person's body, the method may involve (1) turning on the heated air system, (2) allowing enough time to pass for the heated air system to heat air up to a desired temperature (e.g., about 5 minutes), (3) testing the heated air generated by the heated air system with a handheld infrared thermometer to ensure that a desired temperature has been reached, and (4) setting the airflow rate of the heated air system to a desired air flow volume/rate (e.g., 130 cfm or higher or 135 cfm or higher). In one embodiment, heated air is never applied to hair or scalp until after the temperature of the heated air has already reached a desired temperature (e.g., a temperature of at least 100° F., 110° F., 120° F., 130° F., 140° F., 150° F., 155° F., or 160° F. or higher depending on the desired use and desired temperature), and this temperature has been first verified with a thermometer or temperature measuring device. Giving the heated air system time to heat up before use and testing the temperature (e.g., with a thermometer/thermocouple) before use on an animal or person, as may always done, can ensure that heated air below the desired temperature is not used on an animal or person. After these steps, the methods described above of applying the treatment to the animal or person may be used.

In one embodiment, before any heated air is applied to any portion of an animal's or person's body, the method involves (1) turning on the heated air system and setting the device to a high heat setting, (2) allowing at least 5 minutes to pass for the heated air system to heat up, (3) testing the heated air generated by the heated air system with a thermometer to ensure that a desired temperature of the air has been reached (e.g., is at least 150° F. or higher, 155° F. or higher, or 160° F. or higher in one embodiment), and (4) setting the airflow rate to a desired airflow rate (e.g., 130 cfm or higher or 135 cfm or higher). After these steps, the one or more of the methods and/or steps described above may be used, including applying the treatment to the animal or person to be treated as described.

The system, devices/apparatuses, applications, methods, etc. discussed herein may be used to dehydrate and thereby render non-viable and eliminate any remaining eggs (e.g., small eggs) after ectoparasites (e.g., hatched ectoparasites, nymphs, lice, etc.) and/or large eggs have been removed. The following components/aspects/steps may be used: a heated forced air system (including heater/blower device producing heated air, hose, nozzle, stand, and/or other features described herein), a non-toxic lice treatment product (e.g., shampoo, mousse, or spray), a microgrooved, metal nit comb, magnification (e.g., magnifying glass and or magnifying goggles) for final “nit picking” step, any materials or components described herein, and/or other materials. These components may be used together with the methods described herein to eliminate 100% or effectively 100% of lice and nits off a single human head.

In one embodiment, the method may include applying a non-toxic lice treatment product (e.g., a mixture of conditioner, and/or oil, and/or water, or other enzyme-based foams, mousses, shampoos or sprays) that is designed to kill and/or limit or prevent mobility of hatched lice (adults or nymphs (recently hatched lice). In one embodiment, this involves saturating the scalp with a non-toxic lice killing enzyme in one of the forms described above, e.g., using the product Absolute Clear Head Treatment Spray, which is extremely effective and dries easily in the hair without needing to be washed out. This can be an initial or first step or can be a step subsequent to other steps described herein. Performing this step prior to heat treatment helps ensure that hatched lice will not be blown on either the person performing the treatment or the person or animal receiving the treatment.

Another step of the method may include combing the entire scalp with a micro-grooved nit comb designed to remove all hatched ectoparasites (e.g., lice, nymphs, etc.) and as many eggs or nits as possible from the scalp. This may be the same as or similar to other combing steps described elsewhere herein. To prepare for combing, the hair may be sectioned into multiple sections as described elsewhere herein. In one embodiment, the hair is sectioned into four quadrants by making a middle part from forehead to nape of neck, then, a part from ear to ear. The method may include twisting and wrapping each quadrant/section of hair, and clipping the hair to hold it in the quadrant/section. The method may further include using a Nit Free Terminator Comb, comb each quadrant/section, until no hatched ectoparasites or lice are remaining. The majority of eggs or nits may also be removed in this manner, especially relatively larger eggs or nits. If any small or very small eggs are able to avoid removal by the comb, these can be dehydrated and rendered non-viable in a subsequent heated air treatment step. The combing step can occur at different points and before or after other steps, but generally occurs after the step of applying a lice treatment product to kill or slow down the hatched ectoparasites, so they cannot effectively avoid the comb.

Another step may include applying a heated air treatment to the hair to kill any remaining eggs or nits. This step may occur after combing has removed all hatched ectoparasites and many, if not all, of the eggs. Because only eggs or nits remain, lower temperatures can be effectively used to dehydrate any remaining eggs or nits. Any remaining eggs or nits are likely very small and will dehydrate easily as dry air passes over and around them. In some cases, merely passing air through the hair without heating the air may be sufficient to dehydrate and render non-viable any remaining small eggs or nits. Optionally, the air may be heated to between 100° F. and 135° F., between 100° F. and 129° F., between 105° F. and 127° F., between 110° F. and 125° F., between 115° F. and 125° F., or between 120° F. to 125° F. The lower temperatures may be especially effective at dehydrating eggs or nits at a comfortable temperature for the user and person being treated after hatched ectoparasites have been removed (e.g., by combing and other steps described herein). These lower temperatures might also be effective for dehydrating hatched ectoparasites, for example, if the air is blown for a sufficient amount of time at the hatched ectoparasites. The methods/steps of sectioning the hair for heat treatment and applying the heated air treatment described elsewhere herein may also be used in this step.

In one embodiment, the heated air treatment step may include one or more or all of the following: (1) making sure the scalp is dry, prior to using the forced, heated air system or heater/blower device; (2) turning the forced, heated air system using the controls, e.g., setting the controls to the desired heat setting, air flow rate setting, and/or anion setting; (3) sectioning the hair (e.g., in the same way as any of the sectioning steps described elsewhere herein) into sections or quadrants; and (4) applying heated air to the sections or quadrants. Applying the hair to the sections or quadrants may be done in a variety of ways and orders. In one embodiment, one may begin at the back, left quadrant, move to the front left quadrant, move to the back right quadrant, and then finish with the front right quadrant. Although other orders are possible. Each section or quadrant can be treated in subsections or areas (e.g., 2, 3, 4, or more subsections or areas). The subsections or areas may be similar to the partially overlapping areas of treatment described elsewhere herein. In one embodiment each section or quadrant is treated in three subsections or areas within the section or quadrant, e.g., the subsections may be (1) nape of neck (for front quadrant, nozzle is placed above the ear at the hairline); (2) center of head (between crown of head and nape of neck), and (3) crown of head. These subsections or areas may partially overlap. The method may include positioning the nozzle horizontally at the nape of the neck. The nozzle (e.g., nozzle teeth) can be held a desired distance from the head of the person being treated, and an extender similar to that shown in FIG. 4A may be used to help ensure the nozzle is held the correct distance from the head. The method may include “floating” the nozzle/nozzle teeth a desired distance above the head, e.g., 0.5 inches, 1 inch, 1.5 inches, or other distances above the scalp. The method may include moving the nozzle in an “up and down” motion for 30, 45, or 60 seconds or other time intervals as discussed elsewhere herein. This process may be repeated for each subsection in a quadrant/section. Once all subsections have been completed, the hair of the quadrant/section completed may be clipped again. This process may be repeated in each quadrant/section and each subsection within the quadrants/sections. In one embodiment, the treating the sections/quadrants of the hair proceeds in the same or a similar way to other sectioning and heat treating steps/methods described herein.

In addition to heat treating the quadrants as described herein, the method may include treating the central part (e.g., a part along a center of the head from forehead to neck) or other parts in the hair. Three to four or more applications of heated air may be done along the central part or other parts. For example, the central part may be treated in the following treatment areas along the center part: (1) forehead-directly at the hairline; (2) Crown of head (if treatment is done on a larger head, an additional section may be needed to completely cover the crown of the head); (3) Nape of neck (this may involve turning the nozzle upside down to ensure hitting the nape of neck with heated air). The nozzle may be positioned directly over the center part (e.g., parallel with the part) with the tip of the nozzle directed at the hairline (at the nape of neck). The nozzle may be “floated” over the head and moved in a “side to side” motion for 30 seconds. This process may be repeated until the entire center part is treated. Each area or subsection should overlap to ensure 100% coverage.

The method may also include a “nit-picking” step (e.g., going through entire scalp, with magnification, in very thin or paper thin sections) done to make sure that virtually all of lice and eggs/nits are removed from the hair and scalp. This can be done the same as or similar to other nit-picking steps described elsewhere herein. In one embodiment, the nit-picking step may also include putting on a magnification visor or magnification goggles (e.g., Optivisor #5 2.5×) and/or a headlamp. This may also include using a “rat tail” comb to go through the hair in very thin sections. If any stray eggs/nits have been missed in the other steps and remain, the eggs/nits may be removed manually by sliding them off the hair.

Finally, after all other steps, the hair may be put up in a braid or bun, and misted with an ectoparasite prevention spray, e.g., Absolute Clear Prevention Spray.

The above systems, assemblies, devices/apparatuses, methods, etc. have generally been described as being applied to treating head lice infestations; however, the principles described may be applied to other types of infestations. Further, the features described in one embodiment may generally be combined with features described in other embodiments.

While the invention has been described in terms of particular variations and illustrative figures, those of ordinary skill in the art will recognize that the invention is not limited to the variations or figures described. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. Therefore, to the extent there are variations of the invention, which are within the spirit of the disclosure or equivalent to the inventions found in the claims, it is the intent that this patent will cover those variations as well.

What is claimed is:

1. A method of treating a person comprising:
applying a topical product to hair of an animal or person to kill or slow down any hatched ectoparasites on the head;
dividing hair of a person into multiple isolated sections;
combing a first isolated section of hair of the multiple isolated sections with a comb until all hatched ectoparasites are removed from the first isolated section of hair;
applying heated air through a nozzle of a heated air system to the first isolated section of hair at a temperature and air flow sufficient to dehydrate any eggs in the first isolated section; and
picking though the first isolated section in very thin subsections of hair doing a visual check for any remaining eggs and, if found, pulling the remaining eggs off the hair.

2. The method of claim 1, further comprising:
combing a second isolated section of hair of the multiple isolated sections with the comb until all hatched ectoparasites are removed from the second isolated section of hair;
applying heated air through the nozzle of the heated air system to the second isolated section of hair at a temperature and air flow sufficient to dehydrate any eggs in the second isolated section;
picking though the second isolated section in very thin subsections of hair doing a visual check for any remaining eggs and, if found, pulling the remaining eggs off the hair.

3. The method of claim 2, further comprising:
combing a third isolated section of hair of the multiple isolated sections with the comb until all hatched ectoparasites are removed from the third isolated section of hair;
applying heated air through the nozzle of the heated air system to the third isolated section of hair at a temperature and air flow sufficient to dehydrate any eggs in the third isolated section;
picking though the third isolated section in very thin subsections of hair doing a visual check for any remaining eggs and, if found, pulling the remaining eggs off the hair.

4. The method of claim 3, further comprising:
combing a fourth isolated section of hair of the multiple isolated sections with the comb until all hatched ectoparasites are removed from the fourth isolated section of hair;
applying heated air through the nozzle of the heated air system to the fourth isolated section of hair at a temperature and air flow sufficient to dehydrate any eggs in the fourth isolated section;
picking though the fourth isolated section in very thin subsections of hair doing a visual check for any remaining ectoparasites or eggs and, if found, pulling the remaining eggs off the hair.

5. The method of claim 1, wherein applying heated air through the nozzle of the heated air system to the first isolated section of hair includes applying the heated air to a first area within the first isolated section for first time interval sufficient to dehydrate any eggs in the first area, then moving the nozzle to a second area within the first isolated section, wherein the second area overlaps the first area, and then applying the heated air to the second area for second time interval sufficient to dehydrate any eggs in the second area.

6. The method of claim 5, wherein the first time interval and the second time interval are each between 30 and 90 seconds.

7. The method of claim 5, wherein the first time interval and the second time interval are each between 50 and 70 seconds.

8. The method of claim 5, wherein the second area overlaps the first area by between 25-65%.

9. The method of claim 5, wherein the second area overlaps the first area by 45-55%.

10. The method of claim 5, wherein applying heated air through the nozzle of the heated air system to the first isolated section of hair further comprises moving the nozzle to a third area within the first isolated section, wherein the third area overlaps the second area, and then applying the heated air to the third area for a time sufficient to dehydrate any eggs in the third area.

11. The method of claim 1, wherein applying heated air through the nozzle of the heated air system to the first isolated section of hair includes applying the heated air sequentially to multiple areas within the first isolated section for a time sufficient to dehydrate any eggs in each of the multiple areas until the first isolated section has been entirely treated with heated air, and wherein adjacent areas overlap.

12. The method of claim 1, wherein when applying heated air through the nozzle of the heated air system to the first isolated section of hair, the nozzle is held a majority of the time at about 0.15 to 0.35 inches away from the scalp.

13. The method of claim 1, wherein when applying heated air through the nozzle of the heated air system to the first isolated section of hair, hair of the first isolated section is allowed to “parachute” or expand allowing the heated air to be directed through the hair, to the base of the hair and onto the scalp.

14. The method of claim 1, wherein the topical product is non-toxic to humans.

15. The method of claim 1, wherein the comb is a microgrooved metal nit comb.

16. The method of claim 1, wherein the picking step is done using magnification to better see any remaining ectoparasites or eggs.

17. The method of claim 1, wherein the nozzle is kidney shaped and has a concave treatment end.

18. The method of claim 17, wherein the nozzle includes extending portions that extend from the treatment end and can pass through and separate portions of a person's hair.

19. The method of claim 18, wherein the extending portions are removably attachable to the nozzle.

20. The method of claim 19, wherein the nozzle includes a gripping member on a back end of the nozzle, the back end being opposite the treatment end.
